(19) United States
(12) Patent Application Publication
(10) Pub. No.: US 2006/0142555 A1
(43) Pub. Date: Jun. 29, 2006

(54) Process for production of steviosides from stevia rebaudiana bertoni

(75) Inventors: Kotesh K. Jonnala, Palampur (IN); Babu Garikapati D. Kiran, Palampur (IN); Vijay K. Kaul, Palampur (IN); Paramvir S. Ahuja, Palampur (IN)

Correspondence Address: DARBY & DARBY P.C., P. O. BOX 5257, NEW YORK, NY, 10150-5257, US

(73) Assignee: Council of Scientific and Industrial Research, New Delhi (IN)

(21) Appl. No.: 11/022,277

(22) Filed: Dec. 23, 2004

(51) Int. Cl. C07H 15/24

(52) U.S. Cl. 536/18.1

(57) ABSTRACT

A process for the production of steviosides from Stevia rebaudiana Bertoni includes extraction of comminuted plant material by directly injecting steam into the extractor followed by filtration to get aqueous extract and alkali treatment to remove unwanted compounds in the form of precipitate. The treated aqueous extracted was filtered and the filtrate was first treated with gel or macroporous strong acid cation exchange resin and then with gel or macroporous weak base anion exchange resin. The aqueous eluant containing steviosides was concentrated to obtain purified steviosides.

### FIELD OF THE INVENTION

The present invention relates to a process for the production of steviosides from Stevia rebaudiana Bertoni. The present invention relates to a method for recovering steviosides from dried plant material of Stevia rebaudiana using ecofriendly extraction and purification procedures. Water for extraction of the plant material and the ion exchange resins used in the present invention are ecofriendly, non-toxic in nature and are reusable which will ultimately have a tremendous impact on the process economics. The yield of product obtained in the present invention is high and intensity of its sweetness is superior to the plant raw material with milky white in appearance.

### BACKGROUND OF THE INVENTION

Reference is made to R. H. Giovanetto, Method for the recovery of steviosides from plant raw material; U.S. Pat. No. 4,892,938; Jan. 9, 1990 wherein the method involves recovering steviosides from dried plant material of Stevia rebaudiana Bertoni by extraction and purification. An extract is obtained through treatment in water at a temperature from room temperature to about 65° C. with stirring and subsequent filtration and centrifugation. This extract is treated with calcium hydroxide, whereupon a precipitate is obtained by means of filtration or centrifugation. This precipitate is treated with a strongly acidic ion exchange resin and subsequently with a weakly basic ion exchange resin, for three to five times, filtered and then dried. The major drawbacks are that the precipitate was further used to produce steviosides by passing through strongly acidic ion exchange resin and subsequently with a weakly basic ion exchange resin at least for three to five times, which will not only increase the processing time but also affect the overall cost of production. The overall yield of the end product could be low as the steviosides were recovered only from the precipitate instead from the filtrate and this fact was not mentioned any where in the patent specification.

Reference is made to J. D. Payzant; J. K. Laidler; Method of extracting selected sweet glycosides from the Stevia rebaudiana plant; U.S. Pat. No. 5,962,678; Oct. 5, 1999 wherein the method involves production of individual sweet glycosides. A mixture of sweet glycosides extracted from the Stevia rebaudiana plant is processed to remove impurities by using two ion exchange columns. After removing the mixed sweet glycosides from the second column with methanol, the solution is dried. Upon refluxing the dried solids in a methanol solution and then cooling the solution, stevioside precipitates out. The filtrate is further concentrated and cooled to precipitate out Rebaudioside A. This Rebaudioside A can be further purified as the previously obtained Stevioside. The major drawbacks are that the solvent like methanol, which was used for extraction of the steviosides and precipitate steviosides, is harmful to humans and not recommended in the food products all over the world. The steviosides extracts are passed through ion exchange resins without charging the feed and will not give good results and, therefore, this step is more time consuming with low yields. The adsorbent resins used in this process are not advantageous until the stevioside solution is pretreated to remove the metal chelating and colouring impurities. Moreover, the stevioside solutions are needed to be eluted very slowly through these adsorbent resins. Therefore, it is again a time consuming step and usage of many resin columns again contribute to the loss of steviosides. Removal of solvents and re-dissolution of steviosides consumes a lot of energy; heating effect increases the darkening of the colouring impurities and also affects the overall production economics.

Reference is made to Uenishi Hideaki et al., Purification of Stevia sweetening agent; Japan Patent 54030199; 6^{th} Mar., 1979 wherein the process involves to prepare stevia sweetening agent free from characteristic small and bitter taste, by extracting leaves of Stevia rebaudiana Bertoni with water, treating the extract with a non-polar synthetic adsorbent resin followed by desorption, and further treating with an ion exchange resin, etc. The major drawback is treating the aqueous extract with non polar synthetic adsorbent where the non-polar adsorbent will have less affinity for steviosides and more to less polar non-sweet compounds. Therefore, in this process the steviosides are lost partly in eluted water, and when the adsorbed portion is desorbed in water miscible solvent which has to be concentrated. The removal of solvent needs a lot of energy and time. After solvent removal, the concentrate is redissolved in water and treated with ion exchange resins which will not only increase the time, labour and effect the overall economics but also suffers loss of steviosides due to involvement of various resins.

Reference is made to R. H. Dobberstein and M. S. Ahmed; Extraction, separation and recovery of diterpene glycosides from Stevia rebaudiana plants; U.S. Pat. No. 4,361,697; Nov. 30, 1982 wherein a process for recovering diterpene glycosides from the Stevia rebaudiana plant includes the steps of sequentially extracting plant material with a first solvent of intermediate polarity to extract plant substances which tend to interfere with a liquid chromatographic separation of the glycosides, and then with a second solvent of high polarity to extract glycosides and chromatographically separating the extracted glycosides by introducing them onto a liquid chromatography column having a packing of an oxygen-containing organic stationary phase covalently bonded through a silicon atom to an inorganic support, eluting them with a solvent of polarity higher than that of the first solvent but lower than that of the second solvent, and collecting individually eluate fractions rich in respective glycosides. The major drawbacks are use of a variety of solvents to extract and process steviosides, having different polarities including methanol which is toxic and not approved as food grade solvent. Final purification of steviosides is achieved by loading crude steviosides on a column chromatography using adsorbents like silica gel as a stationary phase and eluting the column with the help of two solvents sequentially running through the column, which is not a commercially viable process.

Reference is made to Sato Toru; Purification of steviosides through extraction; Japan Patent JP57005663; Jan. 12, 1982, wherein an extracted solution of leaves of “stevia” with water or water-containing alcohol is concentrated to 10-50 wt % solid content to give a concentrated solution, to which a salt or a base of calcium, iron, or aluminum is added to give a deposit, which is removed. The remaining concentrated solution is adjusted to pH 5-7 and the prepared precipitate is removed to give an aqueous solution containing stevioside. The aqueous solution is passed through an acidic cation exchange resin and a basic anion exchange resin successively, and stevioside is obtained from the passed solution. In the operation, a water-soluble organic solvent, e.g., ethanol, acetone, etc. is dissolved in the solution having passed through the acidic cation exchange resin or an aqueous solution dissolving water-soluble organic solvent is passed through the basic anion exchange resin through which the aqueous solution containing stevioside has passed, and the stevioside is obtained from the passed solution. The major drawbacks are the removal of water from aqueous extract, two stage successive treatment with basic and then with acid salts and solvent addition. The treatment with ion exchanger resins and solvent removal at the end not only increases the number of processing steps but in turn increases the processing time and energy and also affects the overall economics of the production.

Reference is made to Sampath Kumar; Method for recovery of stevioside; U.S. Pat. No. 4,599,403; Jul. 8, 1986 wherein an improved method for the recovery of stevioside from Stevia rebaudiana Bertoni plants is provided which does not require the use of dangerous chemicals or special separation equipment such as ion exchange or chromatography. In the process, the raw material, preferably in comminuted form is first extracted with water, the resulting aqueous extract is treated with a di- or tricarboxylic acid chelating agent to remove metallic and other impurities as well as to lower the pH to less than about 4. Subsequently, a calcium-containing agent is added to precipitate out other impurities. The aqueous extract is essentially neutralized with an acid and is then subjected to extraction with a water-immiscible solvent. Purified stevioside crystals are then recovered by cooling the water layer obtained from the said solvent extraction step. The major drawbacks are that the aqueous extract is treated first with an acid and then with base and later neutralizing the solution and then treating with water immiscible solvents like n-butanol which will not only leave the salt residues in the aqueous solution but also suffer steviosides losses into n-butanol and also lower the yield of steviosides up on crystallization from concentrated aqueous solution.

Various patent applications published and available, which deal with the separation of individual glycosides like stevioside, rebaudioside-A and also as a whole steviosides mixture from naturally occurring sources either by using organic solvents like methanol, ethanol, butanol etc., for selective recrystallization or using synthetic adsorbents for purification. Some of the closely related patents along with their drawbacks with respect to the present invention are given below:

| | | | Part | | |
|---|---|---|---|---|---|
| Title | Applicant | Publ. No. | used | Method employed | Drawbacks |
| Separation of | Ajinomoto Co | JP56121455 | Leaf | Solvents for | One glycoside is |
| Stevioside and | Inc | | and | crystallization | obtained at a time |
| Rebaudioside A | | | Stalk | | & yields are less. |
| by | | | | | Aqueous methanol- |
| crystallization. | | | | | a hazardous |
| | | | | | chemical- was used. |
| Isolation of | Maruzen Kasei | JP57086264 | Lea | Polymeric adsorbent | Organic solvents |
| principal | KK | | and | resin and solvents for | and synthetic |
| sweetening | | | Stalk | crystallization | adsorbents for |
| component of | | | | | purification lead to |
| stevia | | | | | losses in product |
| | | | | | and presence of |
| | | | | | hazardous |
| | | | | | chemicals in the |
| | | | | | end product. |
| Purification of | Tama | JP57075992 | Leaf | Polymeric adsorbent, | Organic solvents |
| stevioside | Seikagaku KK | | | charcoal for | (methanol, acetone) |
| | | | | depolarization and | and synthetic |
| | | | | solvents for | adsorbents for |
| | | | | crystallization | purification lead to |
| | | | | | losses and |
| | | | | | hazardous solvent |
| | | | | | traces in the end |
| | | | | | product. At the end |
| | | | | | one glycoside was |
| | | | | | obtained |
| Decolorization | Maruzen Kasei | JP55111768 | Foliage | Solvent extraction, | Solvent extraction |
| and purification | KK | | | charged of the feed | and aquefying the |
| of stevia sweet | | | | directly on to anion | extracting and |
| component | | | | exchange resin only | treating with only |
| | | | | | anion exchanger |
| | | | | | will not give |
| | | | | | substantial purity in |
| | | | | | the end product. |
| Purification of | Daikin Ind Ltd | P55092400 | Not | Water immiscible | Organic solvents |
| Stevioside | | | given | solvent and methanol | were used to purify |
| | | | | is used to purify | the stevioside |
| | | | | stevioside | |
| Purification of | Mitsubishi | JP54041898 | Leaves | Synthetic adsorbent | Adsorbent resin and |
| Stevioside | Chem Ind Ltd | | | and anion exchange | solvents are used to |
| | | | | resin | purify stevioside |
| Production of | Dainippon Ink | JP7143860 | Plant/ | Solvent like methanol | Solvents like |
| stevia sweetener | & Chem Inc | | Leaf | is used to purify the | methanol are used |
| | | | | sweetener | to purify the |
| | | | | | sweeteners |
| Purification of | Chisso Corp | JP55120770 | Leaf | Different tin salts | Care is not taken |
| stevioside | | | and | e.g., stannous | about the residual |
| solution | | | Stalk | chloride, stannous | salts and end |
| | | | | sulfate, stannic acid | product is in |
| | | | | etc., are used | aqueous solution |
| | | | | precipitates are | phase |
| | | | | removed to get | |
| | | | | purified steviosides | |
| Purification of | Chisso Corp | JP55138372 | Leaf | Different Ca and Fe | Care is not taken |
| stevioside | | | and | salts are used | about the residual |
| solution | | | Stalk | precipitates are | salts and end |
| | | | | removed to get | product is in |
| | | | | purified steviosides | aqueous solution |
| | | | | | phase |
| Stevioside | Masuyama | JP55007039 | Buds | Solvents like | Toxic chemicals are |
| extracted from | Fumio | | and | chloroform is used to | used |
| stevia | | | leaf | purified stevioside | |
| containing | | | | | |
| sweetener | | | | | |
| Separation and | Sanyo | JP54132599 | Leaf | Non polar synthetic | Steviosides have |
| purification of | Kokusaku Pulp | | | adsorbent and | less affinity to these |
| stevioside | Co Ltd. | | | solvents used | resins and solvents |
| sweetening | | | | | are used to recover |
| | | | | | them. |
| Novel Natural | Morita Kagaku | JP59045848 | Leaf | The extract is | The direct extract |
| Sweetener | Kogyo KK | | | optionally treated | loading on |
| | | | | with ion exchange | adsorbent resin will |
| | | | | resins then with | not give purity |
| | | | | adsorbent resins | results more over |
| | | | | | organic solvents are |
| | | | | | needed for |
| | | | | | desorption/elution |
| | | | | | of product. |
| | | | | | Treatment with ion |
| | | | | | exchange resins |
| | | | | | without charging |
| | | | | | the extract will not |
| | | | | | give much purity |
| | | | | | results. |
| Extraction and | Sanpack KK | JP62166861 | Leaf | The aqueous extract | Residual salts may |
| purification of | | | | is treated with | be present in the |
| sweetener | | | | various salts like | final fraction and |
| component | | | | CaCl₂ and other | toxic CaCl₂is used |
| from dry leaf of | | | | adsorbents to purify | in the purification. |
| stevia | | | | steviosides | |
| A method for | Sanyo | JP51131900 | Not | Liquid-liquid | Organic solvents |
| purifying | Kokusaku Pulp | | given | extraction is used | such as n-butanol |
| stevioside | Co Ltd. | | | | are used |
| Preparation of | Yamada | JP55162953 | Leaf | Aqueous extract is | Acids like H₂SO₄ |
| stevioside | Masami | | | treated with acid and | breaks glycosidic |
| | | | | then with base and | linkages and |
| | | | | filtrate is treated to | solvents like |
| | | | | polyamide resin and | butanol are used to |
| | | | | then extracted with | purify stevioside |
| | | | | butanol | |
| Purifying | Mitsubishi | JP58028247 | Leaf | Aqueous extract is | Residual salts may |
| method of | Acetae KK | | | treated with Ca(OH)₂ | be present in the |
| stevioside | | | | and water soluble | final fraction. |
| solution | | | | polymeric flocculant | |
| | | | | e.g., polyacrylamid | |
| | | | | high polymer to get | |
| | | | | pure steviosides | |
| Preparation of | Mitsubishi | JP58028246 | Leaf | The aqueous extract | Calcium chloride is |
| stevioside | Acetate KK | | | is treated with | a toxic chemical |
| | | | | Ca(OH)₂, CaCl₂ while | and residual salts |
| | | | | blowing CO₂ gas to | may be present in |
| | | | | remove impurities. | the final fraction. |
| Purification | Pacific Chem | KR9007421 | Leaf | The leaves were | Amberlite XAD-7 |
| process of | Ind Co | | | extract with Ca(OH)₂ | treatment is a time |
| stevioside | | | | and water and the | consuming step as |
| | | | | extract was treated | the steviosides are |
| | | | | with Amberlite | recovered into |
| | | | | XAD7 and then with | organic solvent and |
| | | | | ion exchange resins | solvent is removed |
| | | | | to get pure | and re dissolved in |
| | | | | steviosides | water to treat with |
| | | | | | ion exchanger |
| | | | | | resins. Without |
| | | | | | XAD7 also better |
| | | | | | purity results can be |
| | | | | | achieved. |
| Process for | Pairfir | KR9005468 | Leaf | Aqueous extract | Toxic CaCl₂ and |
| extracting and | Chemical Co | | | treated with CaCl₂ | solvent methanol is |
| separating of | Ltd | | | and then Amberlite | used along with |
| sweetening | | | | XAD-7 and eluted | column |
| materials from | | | | steviosides with | chromatography |
| stevia | | | | methanol and purified | which is cot |
| | | | | by column | commercially a |
| | | | | chromatography and | viable option. |
| | | | | crystallization by | |
| | | | | methanol | |
| Extraction of | Canada Nat | US5972120 | Plant | Aqueous extract is | Capital investment |
| sweet | Res Council | | | treated with various | on these membrane |
| compounds | | | | membranes like | systems is very |
| from stevia | | | | Microfilter, Ultra | high. |
| rebaudiana | | | | filter and Nano filter | |
| Bertoni | | | | to obtain pure | |
| | | | | compounds | |

### OBJECTS OF THE INVENTION

The main object of the present invention is to provide a process for the production of steviosides from Stevia rebaudiana Bertoni, which obviates the drawbacks as detailed above.

Another object of the present invention is to provide a safer, ecofriendly and economically viable process for the production of steviosides.

Still another object of the present invention is to obtain purified steviosides in a fewer processing steps so as to reduce their time and cost of production.

Yet another object of the present invention is to generate efficient steam extraction methods to recover maximum steviosides from the plant material within shortest time.

Yet another object of the present invention is to use less resin intervention in the process to minimize steviosides losses.

Still another object of the present invention is to eliminate use of volatile and toxic solvents in the production of steviosides by carrying out the entire process using green solvents.

### SUMMARY OF THE INVENTION

Accordingly the present invention provides a process for the production of steviosides from Stevia rebaudiana Bertoni, which comprises drying of plant material at temperature any where from 20 to 50° C., followed by pulverization to 20 to 400 mesh size, extracting the plant material in a solvent consisting of demineralised water and heating the contents by injecting the steam directly or indirectly to a temperature ranging from 50° C. to 120° C. at a steam pressure ranging from 1 to 8 kg/cm² for 0.25 to 4.0 hr, filtering the plant material to obtain an aqueous extract, treating the aqueous extract with basic salt to form a precipitate, filtering the treated extract to separate the precipitate, treating the separated clear filtrate with ion exchange resins and thereafter, drying the treated filtrate to obtain a product containing steviosides having stevioside in the range from 40 to 70% by employing water as the only solvent throughout the process.

In an embodiment of the present invention the plant material was charged into the extraction tank along with or without water.

In another embodiment of the present invention the plant material was dipped in water for 0 (without dipping) to 1 day in the ratio of 1:1 to 1:30.

In yet another embodiment of the present invention extracting the dry or wet plant material by directly injecting steam for 0.25 to 4.0 hr. and the overall temperature of the contents were maintained between 50° C. and 100° C.

In still another embodiment of the present invention extracting the wet plant material by indirectly injecting steam for 0.25 to 4.0 hr. and the overall temperature of the contents were maintained between 50° C. and 100° C.

In another embodiment of the present invention the plant material is heated by directly injecting steam into the extracting tank in a continuous or non continuous mode.

In yet another embodiment of the present invention the plant material is extracted for 1 to 3 times by directly injecting the steam till the ratio of plant raw material to cumulative water extracts falls from 1:10 to 1:30.

In still another embodiment of the present invention the aqueous extract was treated with a basic salt containing divalent cation such as calcium hydroxide or calcium oxide or calcium chloride to form a precipitate.

In another embodiment of the present invention the aqueous extract is basified at pH 8 to 10 to obtain a precipitate.

In yet another embodiment of the present invention the base treated filtrate was optionally treated with neutral alumina to further remove colouring impurities.

In still another embodiment of the present invention the strongly acidic ion exchange resin employed was selected from gel type or macroporous type.

In another embodiment of the present invention the weakly basic ion exchange resin employed was selected from gel type or macroporous type.

In yet another embodiment of the present invention the anion exchange treated steviosides solution was optionally treated with neutral alumina to further remove colouring impurities.

In still another embodiment of the present invention the neutral alumina was washed with plenty of water before treating with steviosides solution.

### DETAILED DESCRIPTION OF THE INVENTION

The present invention provides a process for the production of steviosides from Stevia rebaudiana Bertoni. The process comprises drying of plant material at temperature anywhere from 20 to 50° C., followed by pulverization to 20 to 400 mesh size. The plant material is then extracted in a solvent consisting of demineralised water and the contents heated by injecting steam directly or indirectly to a temperature ranging from 50° C. to 120° C. at a steam pressure ranging from 1 to 8 kg/cm² for 0.25 to 4.0 hr. The plant material is then filtered to obtain an aqueous extract, which is then treated with basic salt to form a precipitate. The treated extract is then filtered to separate a clear precipitate, which is then treated with ion exchange resins and thereafter, dried to obtain a product containing steviosides having stevioside in the range from 40 to 70%. Water is the only solvent employed throughout the process.

The plant material can be charged into the extraction tank along with or without water. Preferably, the plant material was dipped in water for 0 (without dipping) to 1 day in the ratio of 1:1 to 1:30. The dry or wet plant material was extracted by directly injecting steam for 0.25 to 4.0 hr. and the overall temperature of the contents were maintained between 50° C. and 100° C. Alternatively, the wet plant material is extracted by indirectly injecting steam for 0.25 to 4.0 hr. and the overall temperature of the contents were maintained between 50° C. and 100° C.

The plant material is heated by directly injecting steam into the extracting tank in a continuous or non continuous mode. The extraction is effected 1 to 3 times by directly injecting the steam till the ratio of plant raw material to cumulative water extracts falls from 1:10 to 1:30. The aqueous extract was treated with a basic salt containing divalent cation such as calcium hydroxide or calcium oxide or calcium chloride to form a precipitate. The aqueous extract is basified at pH 8 to 10 to obtain a precipitate. The base treated filtrate was optionally treated with neutral alumina to further remove colouring impurities. The strongly acidic ion exchange resin employed was selected from gel type or macroporous type. The weakly basic ion exchange resin employed was selected from gel type or macroporous type. The anion exchange treated steviosides solution was optionally treated with neutral alumina to further remove colouring impurities. Neutral alumina was washed with plenty of water before treating with steviosides solution.

Dry powder of Stevia rebaudiana plant material 20 to 400 mesh size cultivated in the institute farm, was charged into hydro extraction unit along with water in the ratio of 1:0 to 1:10 and heated the contents at 50° to 120° C. for 0.25 to 4.0 hrs by directly or indirectly injecting steam, generated in a separate boiler at a pressure in the range of 1 to 8 kg/cm². The water extract was filtered to get rid off plant particles. The aqueous extract containing colouring matter, steviosides and other compounds was treated with di- or tri-valent salts such as calcium hydroxide or calcium chloride or calcium oxide or aluminum salts or iron salts by maintaining the pH of the solution between 8 and 10 with continuous agitation for 2-5 minutes to obtain the precipitate. The precipitate was separated by filtration and washed with plenty of fresh water. The washings were added to the filtrate and the filtrate was, optionally, treated with neutral alumina. The separated filtrate was passed through a strong cation exchange resin and then through a weak anion exchange resin. The flow rate in both these resins was maintained between 1 to 3 bed volumes per hour. After completely passing, the two resin columns were washed with fresh tap water to recover the steviosides left in the column. The final eluant obtained from anion resin was, optionally, treated with neutral alumina to remove further impurities. The filtrate was dried to obtain purified steviosides.

The dried Stevia rebaudiana plant material was dipped/soaked in minimum amount of water just to wet the plant matrix and heated the contents by directly injecting the steam with a high pressure 2-10 kg/cm² in order to extract the steviosides into the aqueous medium in an efficient way in a short time. The steam, directly injected into the extractor, surrenders its readily available latent heat onto the wet plant tissue so as to enable quick release of water soluble steviosides. This method not only reduces the process time and enhances the extraction efficiency but also helps in reducing the cost of production. In addition, the amount of water required to extract the plant material in a batch is very little compared to the conventional extraction methods. This in turn reduces the cost of the extraction equipment. In the prior arts, the aqueous extract was treated with acid/base to precipitate and the precipitate/filtrate was further purified either with water miscible/immiscible organic solvents or by treating with various synthetic polymeric adsorbent resins/ion exchange resins to obtain steviosides. On the contrary, in the present invention the aqueous extract is treated with an alkali only and the filtrate is directly subjected to gel/macroporous type strong cation exchanger resin and then to gel/macroporous type weak anion exchange resin.

The following examples are given by way of illustration and therefore should not be construed to limit the scope of the present invention.

### EXAMPLE-1

One kilo gram of Stevia plant powder 200 to 400 mesh sizes was extracted with demineralised water (3×4 L) at 70 to 80° C. The extract was cooled and filtered on a Wattman 40 filter paper. The filtrate having about 40% dissolved solids was treated with aqueous calcium hydroxide (50 gm in 50 ml distilled water) till the pH was attained to 9.2 at room temperature and stirred for half an hour to obtain a precipitate. The precipitate was removed by filtration and the clear filtrate was passed through previously washed and equilibriated strong cation exchange resin (Tulsion T-42H⁺ Gel type, 1 liter procured from M/S Thermax Ltd., Pune, India) and then through a weak anion exchange resin (Tulsion A2 XMP Macroporous type, 1 liter procured from M/S Thermax Ltd., Pune, India). The two resin beds were packed into 65 cm×5 cm i.d. glass columns separately. The flow rate in the two resins was maintained at 2 bed volumes per hour. The eluant coming out of anion exchanger column was concentrated to get purified steviosides having 63.4% stevioside in the end product.

### EXAMPLE-2

One kilo gram of Stevia plant powder 200 to 400 mesh sizes was extracted with demineralised water (3×4 L) at 70 to 80° C. The extract was cooled and filtered on a Wattman 40 filter paper. The filtrate having about 40% dissolved solids was treated with aqueous calcium hydroxide (50 gm in 50 ml distilled water) till the pH was attained to 9.2 at room temperature and stirred for half an hour to obtain a precipitate. The precipitate was removed by filtration and the clear filtrate was treated with 250 gm neutral alumina (which was previously washed with 1 liter of fresh water), the contents were agitated for 15 minutes, filtered and then passed through previously washed and equilibriated strong cation exchange resin (Amberlite IRA 120, gel type, M/S Merck India Ltd. Mumbai) and then through a weak anion exchange resin (Amberlite IR 400, gel type, M/S Merck India Ltd. Mumbai). The two resin beds were packed into 65 cm×5 cm i.d. glass columns separately. The flow rate in the two resins was maintained at 2 bed volumes per hour. The eluant coming out of anion exchanger column was concentrated to get purified steviosides having 60% stevioside in the end product.

### EXAMPLE-3

One kilo gram of Stevia leaf powder 200 to 400 mesh sizes was extracted with water (3×4 L) at 70 to 80° C. The extract was cooled and filtered on a Wattman 40 filter paper. The filtrate having about 40% dissolved solids was treated with aqueous calcium hydroxide (50 gm in 50 ml distilled water) till the pH was attained to 9.5 at room temperature and stirred for half an hour to obtain a precipitate. The precipitate was removed by filtration and the clear filtrate was passed through previously washed and equilibriated strong cation exchange resin (Tulsion T-42 MP, macroporous type, 1 liter procured from M/S Thermax Ltd., Pune, India) and then through a weak anion exchange resin (Tulsion A2-XMP macroporous type, 1 liter procured from M/S Thermax Ltd., Pune, India). The two resin beds were packed into 65 cm×5 cm i.d. glass columns separately. The flow rate in the two resins was maintained at 2 bed volumes per hour. The eluant coming out of anion exchanger column was concentrated to get purified steviosides having 65.5% stevioside in the end product.

### EXAMPLE-4

Ten kilogram dry Stevia plant powder from Stevia rebaudiana, cultivated in the Institute's farms, was charged into 300 liter capacity hydro extraction unit along with 45 liters of demineralised water and heated the contents at 70° C. for 2 hrs by directly injecting steam, generated in a separate boiler at a pressure of 4 kg/cm². The water extract was filtered into PVC buckets to get rid off plant particles. 300 liters of this extract containing colouring matter, steviosides and other compounds was treated with aqueous calcium hydroxide (500 grams in 1 liter) by maintaining the pH of the solution at 9.5 with continuous agitation for 4 minutes to obtain precipitate. The precipitate was removed by filtration and then washed with fresh water in order to recover steviosides effectively. These washings were added to the aqueous filtrate and passed through strong cation exchange resin (Tulsion T-42H⁺ gel type, 30 liters procured from M/S Thermax Ltd., Pune) which was earlier equilibriated with 3 bed volumes i.e., 3×10 liters of dil. HCl (1 liter in 9 liter water). This resin was packed in a SS column fitted with a valve at the bottom with a flow rate not exceeding 200 ml/min (1.2 bed volume per hour). The eluant coming out of cation exchange column containing steviosides was again immediately passed through weak anion exchanger (Tulsion A-2×MP 30 Liters, macroporous type, procured from M/S Thermax Ltd., Pune) which was packed in a SS column (earlier equilibriated with 3 bed volumes i.e., 3×12 liters, of NaOH solution; 500 gm in 10 liter water). The flow rate was maintained to not more than 200 ml/min (1.2 bed volumes per hour). After completely passing the aqueous solution, the two resin columns were washed with water to recover the steviosides left in the column. The final eluant obtained from anion exchanger, about 400 lit., was concentrated to obtain purified steviosides having 45.47% stevioside as the end product.

The soaking of dried plant material in minimum amount of water would have softened the plant tissue, which further facilitated easy extraction of steviosides when steam was directly injected in to the extractor. The live steam surrendered its readily available latent heat onto the wet plant tissue, which helped in quick isolation of steviosides through the cell walls. In addition, the directly injected steam create an agitation effect, which helps in instantaneous uniform heat distribution and cell wall rupture throughout the feed material whereby increasing the rate of extraction of steviosides. This method not only reduced the process time and enhanced the extraction efficiency but also helped in reducing the cost of production. The aqueous extract was partially purified by treating only with an alkali to remove various chelating, high molecular weight and colouring compounds in the form of a precipitate. The filtrate separated from precipitate was directly subjected to various combinations of gel and macroporous type ion exchange resins. This not only cut-short the process purification time but also enabled the process to be carried out in an aqueous medium by avoiding intervention of organic solvents and also affecting the cost of production.

The Main Advantages of the Present Invention are:

1. The process is performed with safer and reusable chemicals like water, ion exchange resins in the food grade range and the entire process is carried out in only one solvent phase and that is water.
2. The extraction of the plant material is performed by directly injecting steam where the maximum steviosides are recovered in less time and in little water, which reduces the capital investment on plant machinery.
3. Most of the colouring impurities like chlorophylls, flavonoids and other chelating compounds are precipitated by calcium hydroxide treatment at room temperature by mere stirring.
4. The separated filtrate is further purified by directly subjecting onto ion exchanger resins.
5. The process is simplified by eliminating usage of polymeric adsorbent resins and organic solvents.

We claim:

1. A process for the production of steviosides from Stevia rebaudiana Bertoni, which comprises drying plant material, followed by pulverization to 20 to 400 mesh size, extracting the pulverized plant material in a solvent consisting of demineralised water under heat, filtering the heated plant material to obtain an aqueous extract, treating the aqueous extract with a basic salt to form a clear precipitate, separating the precipitate, treating the separated clear filtrate with an ion exchange resin, followed by drying the treated filtrate to obtain a product containing steviosides having stevioside in the range from 40 to 70%.

2. A process as claimed in claim 1 wherein the plant material is dried at a temperature in the range of 20 to 50° C.

3. A process as claimed in claim 1 wherein the dried plant material is extracted while heating by injecting the steam directly or indirectly to a temperature ranging from 50° C. to 120° C. at a steam pressure ranging from 1 to 8 kg/cm² for 0.25 to 4.0 hr.

4. A process as claimed in claim 1 wherein the precipitate is separated by filtration.

5. A process as claimed in claim 1 wherein the plant material is charged into an extraction tank along with or without water.

6. A process as claimed in claim 1 wherein the plant material is dipped in water for 0 (without dipping) to 1 day in the ratio of 1:1 to 1:30.

7. A process as claimed in claim 1 wherein the dry or wet plant material is extracted directly injecting steam for 0.25 to 4.0 hr. to maintain an overall temperature between 50° C. and 100° C.

8. A process as claimed in claim 1 wherein the wet plant material is extracted by indirectly injecting steam for 0.25 to 4.0 hr. to maintain an overall temperature between 50° C. and 100° C.

9. A process as claimed in claim 1 wherein the plant material is heated by directly injecting steam thereon in a continuous or non continuous mode.

10. A process as claimed in claim 1 wherein the plant material is extracted for 1 to 3 times by directly injecting steam till ratio of plant raw material to cumulative water extracts falls from 1:10 to 1:30.

11. A process as claimed in claim 1 wherein the aqueous extract is treated with a basic salt containing divalent cation selected from the group consisting of calcium hydroxide, calcium oxide and calcium chloride.

12. A process as claimed in claim 1 wherein the aqueous extract is basified at pH 8 to 10 to obtain a precipitate.

13. A process as claimed in claim 1 wherein the base treated filtrate is treated with neutral alumina to remove colouring impurities.

14. A process as claimed in claim 1 wherein the ion exchange resin is a strongly acidic type ion exchange resin and is selected from gel type or macroporous type.

15. A process as claimed in claim 1 wherein the ion exchange resin is a weakly basic ion exchange resin and is selected from gel type or macroporous type.

16. A process as claimed in claim 1 wherein the anion exchange treated steviosides solution is treated with neutral alumina to remove colouring impurities.

17. A process as claimed in claim 16 wherein the neutral alumina is washed with water before treating the stevioside solution.
